(19) United States
(12) Plant Patent Application Publication
(10) Pub. No.: US 2016/0183425 P1
(43) Pub. Date: Jun. 23, 2016

(54) Blackberry Plant Named 'DRISBLACKTWELVE'

(50) ;

(71) Applicant: DRISCOLL STRAWBERRY ASSOCIATES, INC., WATSONVILLE, CA (US)

(72) Inventors: GAVIN R. SILLS, GILROY, CA (US); ANDREA M. PABON, GILROY, CA (US); MARK CRUSHA, CAPITOLA, CA (US)

(21) Appl. No.: 14/544,300

(22) Filed: Dec. 19, 2014

(51) Int. Cl. A01H 5/00

(52) U.S. Cl. PLT/203

(57) ABSTRACT

This invention relates to a new and distinct variety of blackberry plant named ‘DrisBlackTwelve’, particularly characterized by having medium to high vigor, medium-sized, black fruit and medium to high yield, is disclosed.

### GENUS AND SPECIES

Rubus L. subgenus Rubus

### VARIETY DENOMINATION

‘DrisBlackTwelve’

### BACKGROUND OF THE NEW PLANT

The present invention relates to a new and distinct blackberry cultivar designated ‘DrisBlackTwelve’ and botanically known as Rubus L. subgenus Rubus. This new blackberry variety was identified in Santa Cruz County, California in June 2008 and originated from a cross between the proprietary female parent blackberry plant 13M711 (858A5)' (unpatented) and the proprietary male parent blackberry plant ‘BJ111.1’ (unpatented). The original seedling of the new cultivar was first asexually propagated by tissue culture and root cuttings at a nursery in Santa Cruz County, California. ‘DrisBlackTwelve’ was subsequently asexually propagated by tissue culture and root cuttings and underwent further testing at a nursery in Santa Cruz County, California from 2012 to 2014. The present invention has been found to be stable and reproduce true to type through successive asexual propagations.

Plant Breeder's Rights for this variety have not been applied for. ‘DrisBlackTwelve’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Santa Cruz County, California.

1. Medium to high vigor;

2. Medium-sized, black berries; and

3. Medium to high yield.

### DESCRIPTION OF THE PHOTOGRAPHS

This new blackberry plant is illustrated by the accompanying photographs which show the plant's canes, flowers; fruit, and leaves. The colors shown are as true as can be reasonably obtained by conventional photographic procedures. The photographs are of plants that are 2 years old.

FIG. 1 shows a section of a young cane without spines.

FIG. 2 shows the flowers at various stages.

FIG. 3 shows the upper and lower surfaces of the leaves.

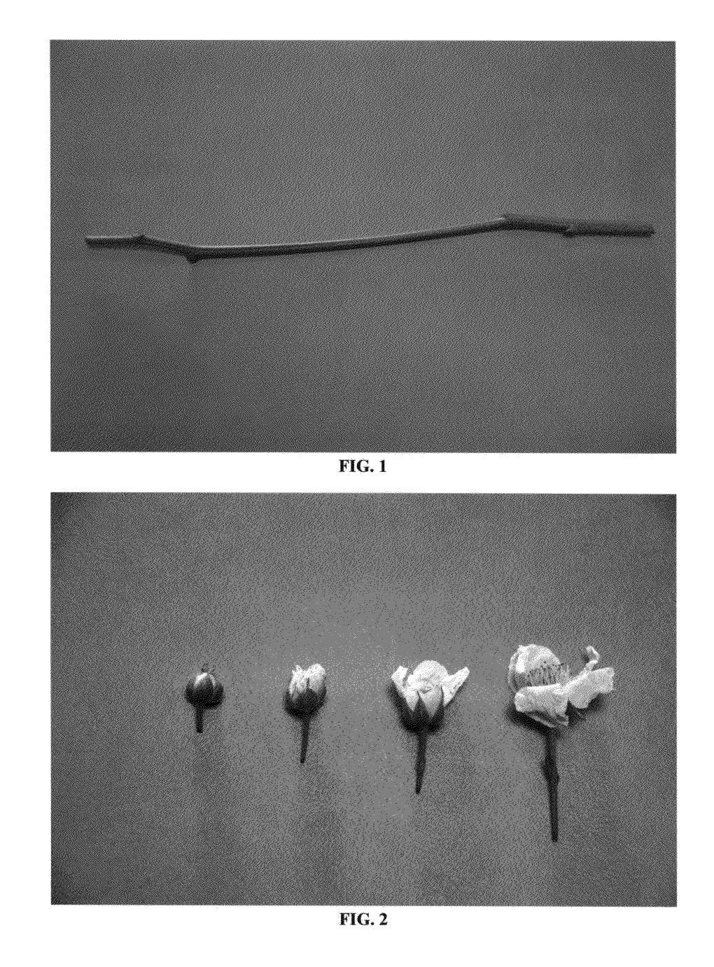

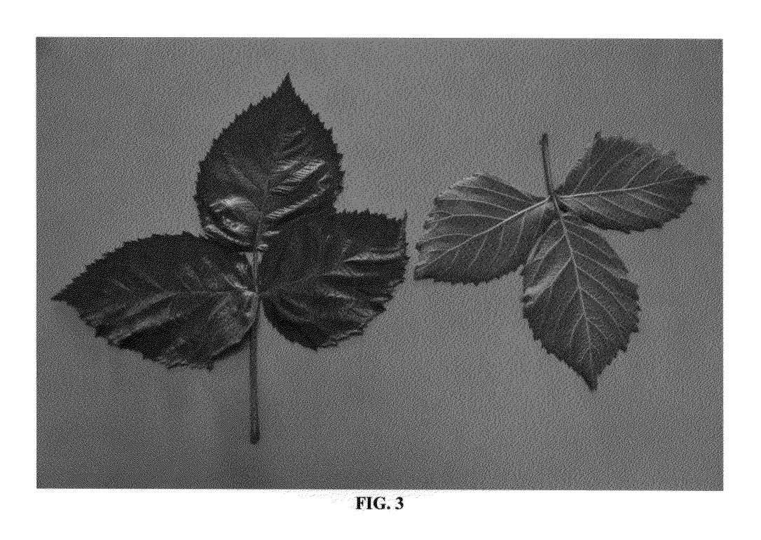

### DESCRIPTION OF THE NEW VARIETY

The following description of ‘DrisBlackTwelve’ are based on observations made in Santa Cruz County, California on 2-year old plants. This description is in accordance with UPOV terminology. Color designations, color descriptions, and other phenotypical descriptions may deviate from the stated values and descriptions depending upon variation in environmental, seasonal, climatic and cultural conditions. ‘DrisBlackTwelve’ has not been observed under all possible environmental conditions. Color terminology follows The Royal Horticultural Society Colour Chart, London (R.H.S.) (2007 edition). Descriptive terminology is from the Plant Identification Terminology, An Illustrated Glossary 2^{nd} edition, by James G. Harris and Melinda Woolf Harris, unless where otherwise defined.

### DETAILED BOTANICAL DESCRIPTION

Classification:
   Family.—Rosaceae.
   Botanical.—Rubus L. subgenus Rubus.
   Common name.—Blackberry.
   Variety name.—‘DrisBlackTwelve’.
Parentage:
   Female parent.—Proprietary blackberry plant named ‘BM711 (858A5)’ (unpatented).
   Male parent.—Proprietary blackberry plant named ‘BJ111.1’ (unpatented).
Plant:
   Vigor.—Medium to high.
   Growth habit.—Upright to semi-upright.
   Productivity.—Medium.
   Propagation.—Tissue culture and root cuttings.
   Self-fruitfulness.—Partially self-fruitful.
   Time of bud burst.—Late.
Canes:
   Fruiting lateral length (4^{th} lateral from tip).—Medium; 34.8 cm.
   Number of fruit per lateral.—12.
   Fruiting on current year's cane.—Present.
   Cane internodal distance.—6.2 cm.
   Number of new canes during flowering.—Few to medium.
   New cane strength (observed full-grown shoot after picking).—Medium.
   Glaucosity observed on full-grown shoot after picking.—Medium.
   Young shoots.—Quantity: Medium Time of emergence from soil: Late Anthocyanin coloration (during rapid growth): Medium; RHS 183A (Dark greyed-purple) Intensity of green color: Medium; RHS 144A (Medium yellow-green) Number of glandular hairs: Medium.
   Dormant cane.—Length: Medium to long Diameter (in central third): Medium Cross section (from mid cane observed at end of first growing season): Rounded to angular Color: RHS 148B (Medium yellow-green) Number of branches: Medium to many Distribution of branches: Only on upper half Anthocyanin coloration: Medium; RHS 143A (Medium green) Spines: Absent.
Leaves:
   Type.—Palmate.
   Predominant number of leaflets.—Five.
   Overlapping or relative position of lateral leaflets.—Overlapping.
   Color.—Upper surface: Dark; RHS 137A (Dark green) Lower surface: Medium; RHS 144B (Medium yellow-green).
   Glossiness of upper side.—Weak to medium.
   Cross section profile.—Convex (margins rolled backwards).
   Leaflets.—Shape: Ovate Margin: Doubly serrate Depth of incisions: Medium Type of incision margin: Bi-serrate Apex: Truncate Base: Obtuse Rachis length between terminal leaflet and adjacent lateral leaflet: 54.6 mm.
   Lateral leaflet (basal pair).—Length: 127.6 mm Width: 89.1 mm Length/width ratio: 1.4.
   Terminal leaflet.—Length: Medium; 139.8 mm Width: Medium; 107.6 mm Length/width ratio: 1.3 Lobing: Absent Undulation of margin (rippling of margin): Medium Blistering between veins (rugosity): Medium Shape: Ovate Tip: Truncate Base: Obtuse Margin: Doubly serrate Cross-section: U-shaped.
   Petiole.—Length: 29.3 mm Diameter: 2.5 mm Pigmentation: Upper surface: Medium; RHS 157C (Green white) Lower surface: Medium; RHS 157C (Green white).
   Stipule.—Size: Medium Orientation: Clasping.
Inflorescence:
   Time of beginning of flowering on previous year's cane.—N/A.
   Time of beginning of flowering on current year's cane.—Late.
   Flower diameter.—Medium to large.
   Flower number (at 3^{rd} node from tip of lateral).—2.
   Petal.—Width: 16.84 mm Length/width ratio: 1.3 Color: RHS 155D (White).
   Pedicel.—Length: 53.47 mm Diameter: 1.56 mm.
Fruit:
   Length.—Medium; 32.66 mm.
   Width.—Medium; 25.30 mm.
   Ratio of length to width.—Medium to large; 1.3.
   Size.—Medium.
   Glossiness.—Medium to strong.
   Shape in longitudinal section.—Medium ovate.
   Weight (g/fruit).—9.94 g.
   Soluble solids (%) (in Brix).—9.3.
   Titratable acidity.—(% as citric acid): 0.91.
   Seed weight (g/seed).—0.005176.
   Number of drupelets/fruit.—Medium; 100.95.
   Size of drupelet.—Medium to large.
   Firmness.—Medium.
   Color.—Immature: RHS 144A (Medium yellow-green) Maturing: RHS 60B (Dark red-purple) Mature: RHS 203A (Black).
   Time of ripening on previous year's cane.—N/A.
   Time of ripening on current year's cane.—Late.
   Harvest interval.—Mid-August-mid-November.
   Yield.—Medium to high.
Disease, pest, and stress resistance: Not tested.

### COMPARISON WITH PARENTAL AND COMMERCIAL VARIETIES

‘DrisBlackTwelve’ differs from the proprietary female parent ‘BM711 (858A5)’ (unpatented) in that ‘DrisBlackTwelve’ is thornless, whereas ‘BM711 (858A5)’ has thorns.

‘DrisBlackTwelve’ differs from the proprietary male parent ‘BJ111.1’ (unpatented) in that ‘DrisBlackTwelve’ has larger fruit and later production than ‘BJ111.1’.

‘DrisBlackTwelve’ differs from the commercial variety ‘DrisBlackNine’ (U.S. Plant patent application Ser. No. 14/121,573) in that ‘DrisBlackTwelve’ has an upright to semi-upright growth habit, palmate type leaves and medium ovate shaped fruit, whereas ‘DrisBlackNine’ has an upright growth habit, intermediate type leaves and oblong shaped fruit. Additionally, ‘DrisBlackTwelve’ has medium to large sized drupelets, whereas ‘DrisBlackNine’ has small to medium sized drupelets.

‘DrisBlackTwelve’ differs from commercial variety ‘DrisBlackTen’ (U.S. Plant patent application Ser. No. 14/121,217) in that ‘DrisBlackTwelve’ has medium productivity and palmate type leaves, whereas ‘DrisBlackTen’ has high productivity and odd-pinnate type leaves. Additionally, ‘DrisBlackTwelve’ has medium to strongly glossy, medium sized fruit, whereas ‘DrisBlackTen’ has medium glossy, large sized fruit.

We claim:

1. A new and distinct variety of blackberry plant named ‘DrisBlackTwelve’, substantially as described and illustrated herein.
